(19) United States
(12) Patent Application Publication
(10) Pub. No.: US 2022/0135667 A1
(43) Pub. Date: May 5, 2022

(54) THERAPEUTIC COMBINATIONS AND COMPOSITIONS FOR THE TREATMENT OF INFLAMMATORY BOWEL DISEASE (II)

(71) Applicants: RIJSUNIVERSITEIT GRONINGEN, Groningen (NL); DSM IP ASSETS B.V., Heerlen (NL); ACADEMISCH ZIEKENHUIS GRONINGEN, Groningen (NL)

(72) Inventors: Gerard DIJKSTRA, Groningen (NL); Klaas Nico FABER, Groningen (NL); Hermanus Jozef Martinus HARMSEN, Groningen (NL); Robert STEINERT, Kaiseraugst (CH); Wilbert SYBESMA, Kaiseraugst (CH); Araksya TOPCHYAN, Kaiseraugst (CH)

(21) Appl. No.: 17/427,823

(22) PCT Filed: Feb. 4, 2020
(86) PCT No.: PCT/EP2020/052650

§ 371 Date: Aug. 2, 2021

(30) Foreign Application Priority Data
Feb. 4, 2019 (EP) 19155308.0
Feb. 4, 2019 (EP) 19155322.1

(51) Int. Cl. C07K 16/24; A61P 29/00; A61K 31/525; A61P 1/00

(52) U.S. Cl. CPC C07K 16/241; A61P 29/00; A61K 2039/505; A61P 1/00; A61K 31/525

(57) ABSTRACT

The present invention relates to pharmaceutical combinations of at least one TNF inhibitor and riboflavin to treat patients suffering from inflammatory bowel disease (IBD) or other inflammatory conditions (such as rheumatoid arthritis, ankylosing spondylitis, psoriasis, lupus erythematous and multiple sclerosis). This invention also relates to additive and/or combinations of at least one TNF inhibitor and riboflavin. This invention is also related to a method for the treatment or prophylaxis of IBD, which method comprises administering a therapeutically effective amount of TNF inhibitor and riboflavin.

The present invention relates to pharmaceutical combinations of at least one TNF inhibitor and riboflavin to treat patients suffering from inflammatory bowel disease (IBD) or other inflammatory conditions (such as rheumatoid arthritis, ankylosing spondylitis, psoriasis, lupus erythematous and multiple sclerosis). This invention also relates to additive and/or combinations of at least one TNF inhibitor and riboflavin. This invention is also related to a method for the treatment or prophylaxis of IBD, which method comprises administering a therapeutically effective amount of TNF inhibitor and riboflavin.

TNF (Tumor Necrosis Factor) inhibitors (also known as TNF alfa inhibitors, TNF-α inhibitors) are a group of medicines that suppress the body's natural response to tumor necrosis factor (TNF), a protein produced by white blood cells that is involved in early inflammatory events.

TNF is involved in autoimmune and immune-mediated disorders such as rheumatoid arthritis, ankylosing spondylitis, inflammatory bowel disease, psoriasis, hidradenitis suppurativa and refractory asthma, so TNF inhibitors may be used in their treatment.

Inhibition of TNF effects can be achieved with a monoclonal antibody such as infliximab (Remicade), adalimumab (Humira), certolizumab pegol (Cimzia), and golimumab (Simponi), or with a circulating receptor fusion protein such as etanercept (Enbrel).

Thalidomide (Immunoprin) and its derivatives lenalidomide (Revlimid) and pomalidomide (Pomalyst, Imnovid) are also active against TNF.

While most clinically useful TNF inhibitors are monoclonal antibodies, some are simple molecules such as xanthine derivatives (e.g. pentoxifylline) and bupropion.

Several 5-HT2A agonist hallucinogens including (R)-DOI, TCB-2, LSD and LA-SS-Az are also inhibitors of TNF.

There is a range of commercially available TNF inhibitors on the market. The most important ones are the following ones (in brackets are the commercial drug forms):
Adalimumab (Humira®)
Certolizumab pegol (Cimzia®)
Etanercept (Enbrel®)
Golimumab (Simponi®)
Infliximab (Remicade®)

Usually all the TNF inhibitors are administered by injection into a vein.

Riboflavin, also known as vitamin B2, is a micronutrient with a key role in maintaining health in humans and other mammals. It is the central component of the cofactors

FAD and FMN, and is therefore required by all flavoproteins. As such, riboflavin is required for a wide variety of cellular processes. It plays a key role in energy metabolism, and for the metabolism of fats, ketone bodies, carbohydrates, and proteins. Moreover, riboflavin has anti-inflammatory and anti-oxidant effects. Riboflavin is found naturally in asparagus, popcorn, bananas, per-simmons, okra, chard, cottage cheese, milk, yogurt, meat, eggs, fish, and green beans. Other sources specify cheese, leafy green vegetables, liver, kidneys, legumes, tomatoes, yeast, mushrooms, and almonds.

Inflammatory Bowel Disease (IBD) is a chronic and debilitating illness. It is characterized by chronic intestinal inflammation that often shows an intermittent course with acute attacks followed by periods of remission. Clinical symptoms during acute attacks include diarrhea, bleeding, abdominal pain, fever, joint pain, and weight loss. These symptoms can range from mild to severe, and may gradually and subtly develop from an initial minor discomfort, or may present themselves suddenly in fullblown form. IBD can manifest itself in a variety of forms, the most common of which are Crohn's disease (CD) and ulcerative colitis (UC). Both of these diseases are similar in terms of clinical symptoms, even though their inflammation patterns are distributed differently in the GI tract. Crohn's disease is a chronic transmural inflammation of the bowel, which can affect the whole gastrointestinal tract, usually in a discontinuous pattern. The initial location of CD is most commonly in the lower ileum. From here the inflammation typically spreads towards proximal parts of the small intestine. However, the colon is also often involved.

Ulcerative colitis is a chronic inflammatory bowel disease affecting only the colon and shows a continuous distribution in the gastrointestinal mucosa. In most patients the focal point of the inflammation is in the distal part of the colon and the rectum. From this origin, the inflammation often spreads proximally. In the most severe cases, the whole colon is affected which is called as “pancolitis”. About 30% of patients suffer from this severe form of UC.

The present invention relates to a pharmaceutical combination (PC) comprising
(i) at least one TNF inhibitor and
(ii) riboflavin.

The present invention relates to a pharmaceutical combination (PC1) consisting of
(i) at least one TNF inhibitor and
(ii) riboflavin.

The present invention relates to a formulation (F) comprises pharmaceutical combinations of
(i) at least one TNF inhibitor and
(ii) riboflavin.

Furthermore, preferably the TNF inhibitor is chosen from the group consisting of adalimumab, certolizumab pegol, etanercept, golimumab and infliximab.

The present invention relates to a pharmaceutical combination (PC′), which is pharmaceutical combination (PC), wherein the TNF inhibitor is chosen from the group consisting of adalimumab, certolizumab pegol, etanercept, golimumab and infliximab.

The present invention relates to a pharmaceutical combination (PC1′), which is pharmaceutical combination (PC1), wherein the TNF inhibitor is chosen from the group consisting of adalimumab, certolizumab pegol, etanercept, golimumab and infliximab.

The present invention relates to a formulation (F′), which is formulation (F), wherein the TNF inhibitor is chosen from the group consisting of adalimumab, certolizumab pegol, etanercept, golimumab and infliximab.

The pharmaceutical combination (PC), (PC′), (PC1) and (PC1′) and the formulation (F) and (F′) are preferably in a liquid form, which can be injected into a vein.

Therefore, the present invention relates to a pharmaceutical combination (PC″), which is pharmaceutical combination (PC) or (PC′), wherein the pharmaceutical combination is in a liquid form (which can be injected into a vein).

Therefore, the present invention relates to a pharmaceutical combination (PC1″), which is pharmaceutical combination (PC1) or (PC1′), wherein the pharmaceutical combination is in a liquid form (which can be injected into a vein).

Therefore, the present invention relates to a formulation (F″), which is formulation (F) or (F′), wherein the formulation is in a liquid form (which can be injected into a vein).

Furthermore, preferably the TNF inhibitor is chosen from the group consisting of adalimumab, certolizumab pegol, etanercept, golimumab and infliximab.

The present invention also relates to a method (M) for the treatment or prophylaxis of IBD or other inflammatory conditions, which method comprises administering a therapeutically effective amount of
(i) at least one TNF inhibitor and
(ii) riboflavin.

The present invention also relates to a method (M′) for the treatment of IBD or other inflammatory conditions, which method comprises administering a therapeutically effective amount of
(i) at least one TNF inhibitor and
(ii) riboflavin.

Another embodiment of this invention is a method of treating or lessening the symptoms of inflammatory bowel disease or other inflammatory conditions comprising administering to a person in need thereof an effective amount of at least one TNF inhibitor and riboflavin.

Therefore, the present invention also relates to a method (M″) of treating or lessening the symptoms of inflammatory bowel disease or other inflammatory conditions comprising administering to a person in need thereof an effective amount of
(i) at least TNF inhibitor and
(ii) riboflavin.

Furthermore, preferably the TNF inhibitor is chosen from the group consisting of adalimumab, certolizumab pegol, etanercept, golimumab and infliximab.

Furthermore the present invention also relates to a method (M″), which is method (M), (M′) or (M″), wherein the TNF inhibitor is chosen from the group consisting of adalimumab, certolizumab pegol, etanercept, golimumab and infliximab.

Another embodiment of this invention is the use of the combination of at least one TNF inhibitor and riboflavin to treat or lessen the symptoms of IBD or other inflammatory conditions.

Therefore, the present invention also relates to the use (U) of the combination of
(i) at least one TNF inhibitor and
(ii) riboflavin

to treat or lessen the symptoms of IBD or other inflammatory conditions.

Another embodiment of this invention is the use of the combination of riboflavin and at least one TNF inhibitor thereof in the manufacture of a pharmaceutical to treat or lessen the symptoms of IBD.

Therefore, the present invention also relates to the use (U′) of the combination of
(i) at least one TNF inhibitor and
(ii) riboflavin

in the manufacture of a pharmaceutical to treat or lessen the symptoms of IBD or other inflammatory conditions.

Furthermore the present invention also relates to a use (U″), which is use (U), or (U′), wherein the TNF inhibitor is chosen from the group consisting of adalimumab, certolizumab pegol, etanercept, golimumab and infliximab.

The combination of at least one TNF inhibitor and riboflavin results in a synergistic effect.

Alternatively it is also possible due to the effect of these two compounds to lower the amount of at least one TNF inhibitor. This is great and surprising effect due to known the side effects of the TND inhibitors (such coughing, headaches, heartburn, nausea or vomiting, stomach pain and weakness).

Therefore the present invention relates to the preventing and/or lessening the side-effects of TNF inhibitors by administering a combination of at least TNF inhibitor and riboflavin to a patient.

Furthermore, a commonly known issue of the TNF inhibitors is that the overall low response rate.

When using the pharmaceutical combination (PC), (PC′) (PC″), (PC1), (PC1′) or (PC1″) or the formulation (F), (F′) or (F″) the response rates are improved surprisingly and significantly.

Therefore the present invention also relates to the use of a pharmaceutical combination (PC), (PC′) (PC″), (PC1), (PC1′) or (PC1″) or of a formulation (F), (F′) or (F″) to improve the response rates.

Furthermore, the present invention also relates to the pharmaceutical combination (PC), (PC′), (PC″) (PC1), (PC1′) or (PC1″) for the use as medicament.

Furthermore, the present invention also relates to the pharmaceutical combination (PC1), (PC1′), (PC1″), (PC1′″) or (PC1′″) for the use as medicament.

Furthermore, the present invention also relates to the formulation (F), (F′) or (F″) for the use as medicament.

Furthermore, the present invention also relates to the pharmaceutical combination (PC), (PC′), (PC″) (PC1), (PC1′) or (PC1″) for use in the treatment of IBD or other inflammatory conditions (especially Crohn's disease).

Furthermore, the present invention also relates to the formulation (F), (F′) or (F″) for use in the treatment of IBD or other inflammatory conditions (especially Crohn's disease.

When using the method for the treatment or prophylaxis of IBD or other inflammatory conditions as disclosed above the sequence of administering the TNF inhibitor and riboflavin can vary. It is possible that first the TNF inhibitor is administered and then the riboflavin (or vice versa). It also possible to administer them together (such as i.e.

in one galenical formulation if that is possible. This formulation is then injected into a vein). It can also be that the sequence can be TNF inhibitor, riboflavin, TNF inhibitor etc. What is meant is that the sequence of administering can vary.

Also in view of the period of time of the administering in case the two compounds are not administered at the same time. This means there can be a gap of time between the intake of the TNF inhibitor and then the riboflavin (or vice versa).

Preferably, TNF inhibitor and riboflavin are the sole active ingredients in the formulation. This means that other (auxiliary) ingredients may be present in the formulation, which are needed for this specific formulation. These (auxiliary) ingredients are usually added to get a suitable and stable formulation (for the injectable formulation).

Therefore, the present invention also relates to a formulation (F1), which is formulation (F), (F′) or (F″), wherein the at least one TNF inhibitor and riboflavin are the sole active ingredients in the formulation.

Therefore, the present invention relates to a formulation (F2) comprising a pharmaceutical combination consisting of the following active ingredients:
(i) at least one TNF inhibitor and
(ii) riboflavin.

Therefore, the present invention also relates to a formulation (F3′), which is formulation (F2), comprising a pharmaceutical combination consisting of the following active ingredients:
(i) adalimumab and
(ii) riboflavin.

Therefore, the present invention also relates to a formulation (F3″), which is formulation (F2), comprising a pharmaceutical combination consisting of the following active ingredients:
(i) certolizumab pegol and
(ii) riboflavin.

Therefore, the present invention also relates to a formulation (F3′″), which is formulation comprising a pharmaceutical combination consisting of the following active ingredients:
(i) etanercept and
(ii) riboflavin.

Therefore, the present invention also relates to a formulation (F3″″), which is formulation comprising a pharmaceutical combination consisting of the following active ingredients:
(i) golimumab and
(ii) riboflavin.

Therefore, the present invention also relates to a formulation (F3″″′), which is formulation comprising a pharmaceutical combination consisting of the following active ingredients:
(i) infliximab and
(ii) riboflavin.

A further embodiment of the present invention is to prepare a formulation (F), (F′), (F″), (F1), (F2), (F3), (F3′), (F3″), (F3′″), (F3″″) and/or (F3″″ ).

These pharmaceutical combinations and/or formulations can be used as such, as a premix as well in any suitable galenical formulation, which can be injected into a vein

Therefore, the present invention also relates to a galenical formulation (GF) comprising a formulation F), (F′), (F″), (F1), (F2), (F3), (F3′), (F3″), (F3″′), (F3″″) and/or (F3″″′).

A further embodiment of the present invention is treatment or lessening of IBD or other inflammatory conditions by the administration a formulation F), (F′), (F″), (F1), (F2), (F3), (F3′), (F3″), (F3″′), (F3″″) and/or (F3″″′).

A further embodiment of the present invention is the treatment of IBD or other inflammatory conditions by the administration a galenical formulation (GF).

As stated above the present invention also related to a method (M) for the treatment or prophylaxis of IBD or other inflammatory conditions, which method comprises administering a therapeutically effective amount of
(i) at least one TNF inhibitor and
(ii) riboflavin.

Once a day means the dosage form(s) to be taken only one time in 24 hours by which the drug concentration is maintained for whole day in the body.

Several time a day means the dosage form(s) to be taken several times in 24 hours.

The galenical formulation can comprise any pharmaceutically acceptable auxiliary agents, which are necessary, needed or desired to form such a galencial formulation.

The galencial formulation can be in any form, which is suitable for a patient to be injected. Typical and commonly used pharmaceutically acceptable excipients for an injectable formulation are citric acid monohydrate, sodium phosphate monobasic dihydrate, dibasic sodium phosphate dihydrate, mannitol, monobasic sodium phosphate dihydrate, polysorbate 80, sodium chloride, sodium citrate, sodium acetate, sucrose, L-arginine hydrochloride, L-hystidine, L-hystidine monohydrochlorate monohydrate, sodium phosphate and purified water.

Therefore, the present invention also relates to a galenical formulation (GF′) comprising a formulation F), (F′), (F″), (F1), (F2), (F3), (F3′), (F3″), (F3′″), (F3″″) and/or (F3″″′), which is an injectable formulation, comprising at least one pharmaceutically acceptable excipient chosen from the group consisting of citric acid monohydrate, sodium phosphate monobasic dihydrate, dibasic sodium phosphate dihydrate, mannitol, monobasic sodium phosphate dihydrate, polysorbate 80, sodium chloride, sodium citrate, sodium acetate, sucrose, L-arginine hydrochloride, L-hystidine, L-hystidine monohydrochlorate monohydrate, sodium phosphate and purified water

By “pharmaceutically acceptable” is meant a material that is not biologically or otherwise undesirable.

When the TNF inhibitor is injected and the riboflavin is taken orally, the riboflavin can in any usually used galenical formulation. (tablet, powder, liquid, gel, etc).

The galenical formulation can comprise any pharmaceutically acceptable auxiliary agents, which are necessary, needed or desired to form such a galencial formulation.

Pharmaceutically acceptable excipients include but are not limited to binders, diluents, lubricants, glidants and surface-active agents.

The amount of additive employed will depend upon how much active agent is to be used. One excipient can perform more than one function.

Binders include, but are not limited to, starches such as potato starch, wheat starch, corn starch; microcrystalline cellulose; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone and the like and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The pharmaceutical formulation according to the present invention include but is not limited to tablets (single layered tablets, multilayered tablets, MUPS, mini tablets, bioadhesive tablets, caplets, matrix tablets, tablet within a tablet, mucoadhesive tablets, modified release tablets, pulsatile release tablets, timed release tablets), pellets, beads, granules, sustained release formulations, capsules, microcapsules, tablets in capsules and microspheres, matrix formulations, microencapsulation and powder/pellets/granules for suspension.

The galenical formulation of the invention can optionally have one or more coatings such as film coating, sugar coating, enteric coating, bioadhesive coating and other coatings known in the art. These coatings help pharmaceutical formulations to release the drug at the required site of action. In one example, the additional coating prevents the dosage from contacting the mouth or esophagus. In another example, the additional coating remains intact until reaching the small and or large intestine (e.g., an enteric coating). Premature exposure of a bioadhesive layer or dissolution of a pharmaceutical dosage form in the mouth can be prevented with a layer or coating of hydrophilic polymers such as HPMC or gelatin. Optionally, Eudragit FS 30D or other suitable polymer may be incorporated in coating composition to retard the release of the drug to ensure drug release in the colon.

These coating layers comprises one or more excipients selected from the group comprising coating agents, opacifiers, taste-masking agents, fillers, polishing agents, coloring agents, antitacking agents and the like.

The galenical formulations of the invention can be coated by a wide variety of methods. Suitable methods include compression coating, coating in a fluidized bed or a pan and hot melt (extrusion) coating. Such methods are well known to those skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Also, coatings of polymers that are susceptible to enzymatic cleavage by colonic bacteria are another means of ensuring release to distal ileum and ascending colon. Materials such as calcium pectinate can be applied as coatings to dosage form and multiparticulates and disintegrate in the lower gastrointestinal tract, due to bacterial action. Calcium pectinate capsules for encapsulation of bioadhesive multiparticulates are also available.

The pharmaceutical compositions of the present invention can optionally include one or more solubilizers, i.e., additives to increase the solubility of the pharmaceutical active ingredient or other composition components in the solid carrier. Suitable solubilizers for, use in the compositions of the present invention include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. Particularly preferred solubilizers include sorbitol, glycerol, triacetin, ethyl alcohol, SLS, polyethylene glycols glycofurol and propylene glycol. Cyclodextrins polyoxomers, surfactants and like

All formulations as well as the galenical formulation described and disclosed above can be produced by using well-known methods and processes.

Another embodiment of this invention is a method of treating or lessening the symptoms of inflammatory bowel disease or other inflammatory conditions comprising administering to a person in need thereof an effective amount of at least one TNF inhibitor and riboflavin.

Therefore, the present invention also relates to a method (M″) of treating or lessening the symptoms of inflammatory bowel disease or other inflammatory conditions comprising administering to a person in need thereof an effective amount of
(i) at least one TNF inhibitor and
(ii) riboflavin.

When using the method for the treatment or prophylaxis of IBD or other inflammatory conditions as disclosed above the sequence of administering the TNF inhibitor and riboflavin can vary. It is possible that first the TNF inhibitor is administered (by injection) and then the riboflavin (or vice versa). It can also be that the sequence can be TNF inhibitor, riboflavin, TNF inhibitor etc. What is meant is that the sequence of administering can vary.

Also in view of the period of time of the administering in case the two compounds are not administered at the same time. This means there can be a gap of time between taken the t TNF inhibitor and then the riboflavin (or vice versa).

TNF inhibitor can be used in the dose range of about 0.8 mg to about 400 mg and riboflavin can be used in a suitable dose range of 1 mg to 500 mg per day which can be administered daily (or several times a day). weekly (or several times a week), monthly (or several times a month). The dosage regime differs for the various TNF inhibitors.

The dosage vary for the various TNF inhibitor.

For adalimumab the dose ranges of from about 40 mg to about 160 mg, for certolizumab pegol the dose ranges of from about 200 mg to about 400 mg; for etanercept the dose ranges of from about 0.8 mg to about 50 mg; for golimumab the dose ranges of from about 30 mg to about 50 mg; for infliximab the dose ranges of from about 5 mg to about 10 mg.

Once a day means the dosage form(s) to be taken only one time in 24 hours by which the drug concentration is maintained for whole day in the body.

Therefore, the present invention relates to a daily dosage unit (DDU1) comprising 0.8 mg to 400 mg of at least one TNF inhibitor and 1 mg to 500 mg (preferably 30 mg-300 mg) of riboflavin.

Therefore, the present invention relates to a daily dosage unit (DDU2) comprising 40 mg to 160 mg of adalimumab and 1 mg to 500 mg (preferably 30 mg-300 mg) of riboflavin.

Therefore, the present invention relates to a daily dosage unit (DDU3) comprising 200 mg to 400 mg of certolizumab pegol and 1 mg to 500 mg (preferably 30 mg-300 mg) of riboflavin.

Therefore, the present invention relates to a daily dosage unit (DDU4) comprising 0.8 mg to 50 mg of etanercept and 1 mg to 500 mg (preferably 30 mg-300 mg) of riboflavin.

Therefore, the present invention relates to a daily dosage unit (DDUS) comprising 30 mg to 50 mg of golimumab and 1 mg to 500 mg (preferably 30 mg-300 mg) of riboflavin.

Therefore, the present invention relates to a daily dosage unit (DDU6) comprising 5 mg to 10 mg of infliximab and 1 mg to 500 mg (preferably 30 mg-300 mg) of riboflavin.

Once a day means the dosage form(s) to be taken only one time in 24 hours by which the drug concentration is maintained for whole day in the body.

Several time a day means the dosage form(s) to be taken several times in 24 hours.

It may also be possible to have dosage units for 2 days, 3 days, 4 days, 5 days, 6 days or weekly dosage units.

### EXAMPLES

In a prospective clinical intervention study, 70 CD patients were included and divided into two groups with (active) and without (quiescent) evidence of mucosal inflammation (defined by fecal calprotectin (FC) cut-off value: 200 μg/g). Patients received 100 mg riboflavin daily for 3 weeks. Clinical disease activity (Harvey-Bradshaw Index: HBI), inflammatory biomarkers (including interleukin 2) as well as fecal microbial composition (including pathogenic Enterobacteriaceae including E. coli) were analyzed before and after riboflavin intervention.

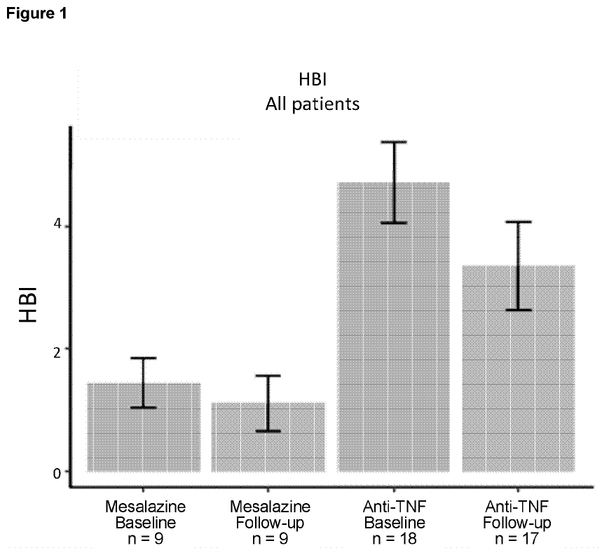

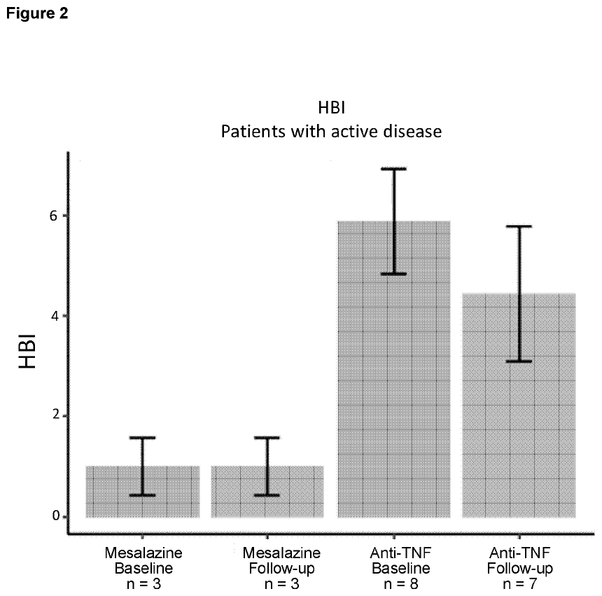

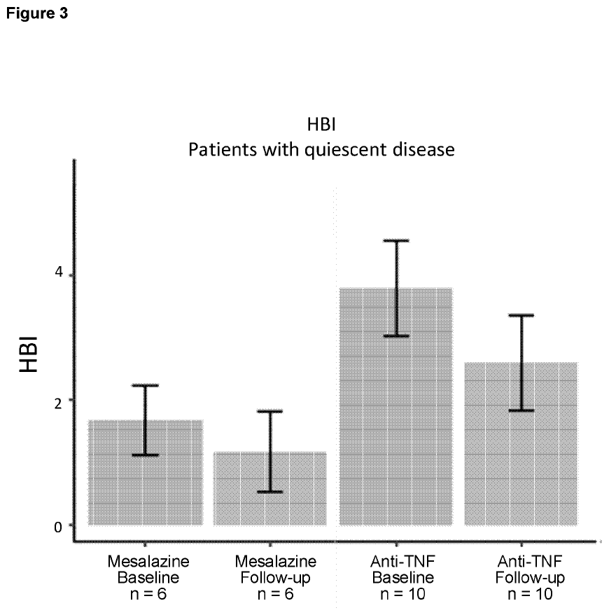

Surprisingly, we found that riboflavin supplementation further reduced clinical disease activity (HBI) in patients with TNF-alpha inhibitor treatment (both in patients with active and quiescent disease), however, this was not the case (not as pronounced as) in patients with mesalazine treatment (FIG. 1-3).

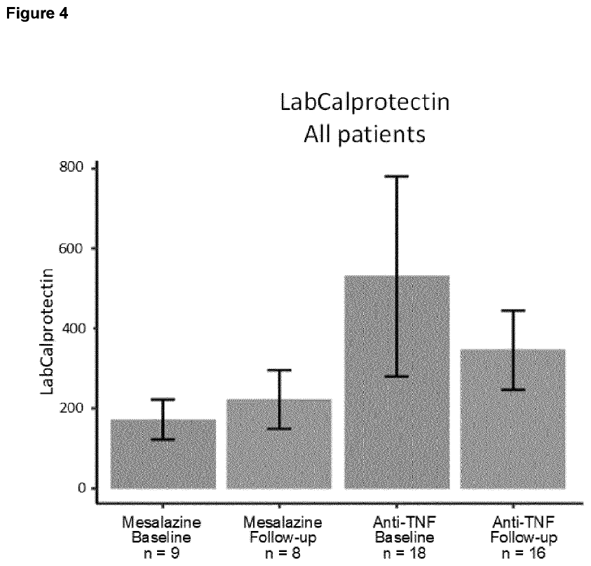

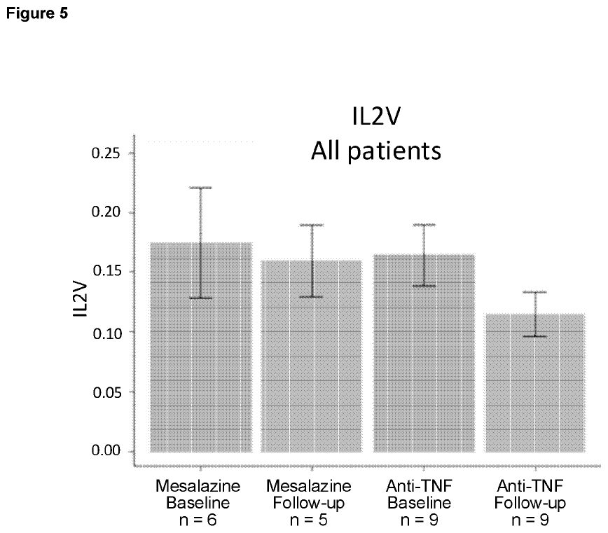

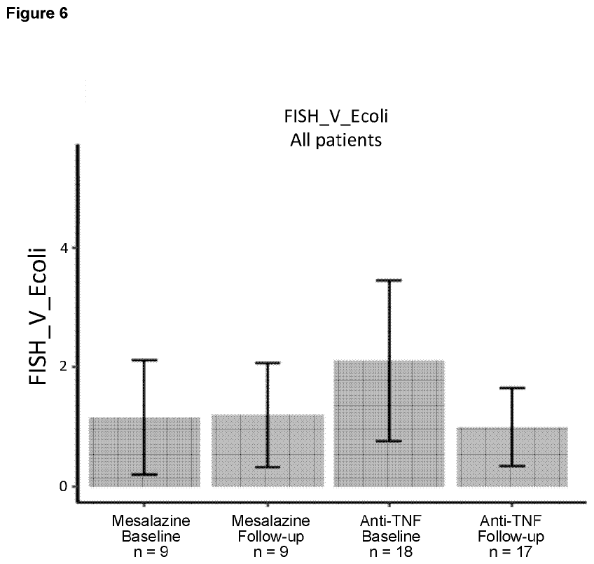

These effects were accompanied by a further reduction in fecal calprotectin (FIG. 4), proinflammatory cytokine IL2 (FIG. 5) as well as Enterobacteriaceae including E. coli (FIG. 6) in patients with TNF-alpha inhibitor treatment, in contrast to patients with mesalazine treatment.

1. A pharmaceutical combination comprising
(i) at least one TNF inhibitor and
(ii) riboflavin.

2. A pharmaceutical combination consisting of
(i) at least one TNF inhibitor and
(ii) riboflavin.

3. Pharmaceutical combination according to claim 1, wherein the TNF inhibitor is chosen from the group consisting of adalimumab, certolizumab pegol, etanercept, golimumab and infliximab.

4. Pharmaceutical combination according to claim 1 wherein the at least one TNF inhibitor and riboflavin are the sole active ingredients in the formulation.

5. A pharmaceutical combination according to claim 1 or a formulation use as therapeutically active composition.

6. Use of the pharmaceutical combination according to claim 1 to treat or lessen the symptoms of IBD or other inflammatory conditions.

7. Pharmaceutical combination according to claim 1 for the use as medicament.

8. Pharmaceutical combination according to claim 1 for use in the treatment of IBD or other inflammatory conditions (especially Crohn's disease).
